(19) United States
(12) Patent Application Publication
(10) Pub. No.: US 2021/0268304 A1
(43) Pub. Date: Sep. 2, 2021

(54) SPONTANEOUS EMISSION TYPE PHOTO CONVERSION SUBSTANCE FOR LIGHT THERAPY, AND THE FUNCTIONAL PATCH AND FUNCTIONAL MASK PACK COMPRISING THE SAME

(71) Applicant: CELLBIOKOREA CO., LTD, Bucheon-si (KR)

(72) Inventor: Sung Hoon LEE, Gimpo-si (KR)

(73) Assignee: CELLBIOKOREA CO., LTD, Bucheon-si (KR)

(21) Appl. No.: 17/098,476

(22) Filed: Nov. 16, 2020

(30) Foreign Application Priority Data
Mar. 2, 2020 (KR) 10-2020-0025734
May 22, 2020 (KR) 10-2020-0061465
Oct. 19, 2020 (KR) 10-2020-0135345

(51) Int. Cl. A61N 5/06; C09K 11/77; C09K 11/64; C09K 11/59; C09K 11/66

(52) U.S. Cl. CPC A61N 5/0616; C09K 11/7774; C09K 11/7766; C09K 11/646; C09K 11/59; A61N 2005/0657; A61N 2005/0663; A61N 2005/0656; A61N 2005/0626; A61N 2005/0645; A61N 2005/0647; C09K 11/662

(57) ABSTRACT

A spontaneous emission type photo conversion substance for light therapy which outputs a specific wavelength, a functional patch, and functional mask pack comprising the same are provided. The photo conversion substance is designed to use a sapphire-based blue axial light fluorescent substance to light exciting in the core, have a relatively long light emission time, and absorb light exciting by selecting a wavelength at the shell part for light emission. The photo conversion substance has a structure capable of selecting a wavelength in order to efficiently express the light therapy function, which is included in the functional patch and functional mask pack.

### CROSS REFERENCE TO THE RELATED APPLICATIONS

This application is based upon and claims priority to Korean Patent Application No. 10-2020-0025734, filed on Mar. 2, 2020, Korean Patent Application No. 10-2020-0061465, filed on May 22, 2020, and Korean Patent Application No. 10-2020-0135345, filed on Oct. 19, 2020, the entire contents of which are incorporated herein by reference.

### TECHNICAL FIELD

This invention pertains to a spontaneous emission type photo conversion substance for light therapy which outputs a specific wavelength, and a functional patch and functional mask pack comprising the same, which more specifically, absorbs sunlight and ambient light to perform a light therapy function, and is also an eco-friendly source of energy of a type close to spontaneous emission under the principle of the form of light emission. Furthermore, it is a substance which has been designed in a complex structure that uses a sapphire-based blue axial light fluorescent substance as exciting light in the core having a relatively long light emission time, and also absorbs exciting light by choosing a wavelength at the shell portion for light emission. It pertains to a photo conversion substance having a structure capable of selecting a wavelength in order to efficiently express the light therapy function, and the functional patch and functional mask pack having the light therapy function comprising the same.

### BACKGROUND

Light therapy uses various artificial light sources which output the wavelength of the visible light area or the near infrared ray area (laser, fluorescent lamp, UV Lamp, etc.), and irradiates the human body with the light of a specific wavelength range, thereby penetrating light energy into the subcutaneous layer of the skin and using the principle of promoting biochemical reactions within cells. It facilitates blood circulation, increases the temperature of the skin, expands blood vessels, facilitates metabolism, helps to regenerate skin cells, and strengthens immunity to relieve pain and inflammation. Hence, it is preferred as a method for treating skin diseases or improving skin conditions, while serving the role of relieving muscle fatigue.

Light therapy is a method of improving or treating skin diseases by selectively using a wavelength of 400 nm to 1,300 nm which exerts a positive effect on the skin among the prescribed wavelengths which are output from the light source, and its scope of use is increasingly expanding.

Examining the verified skin improvement effect by each color wavelength according to the light therapy, a wavelength of 400 nm to 420 nm is effective for the cell growth and regeneration cell regeneration, whereas a wavelength of 440 nm to 500 nm is effective for removing acne bacteria, reducing acne inflammation and controlling sebaceous glands. In addition, a wavelength of 500 nm to 520 nm is effective for soothing irritated skin and reducing capillary dilatation, while a wavelength of 520 nm to 562 nm helps to manage complex skin problems and dark spots, and a wavelength of 565 nm to 590 nm is effective for redness and sunburn caused by inflammation. Furthermore, a wavelength of 590 nm to 620 nm is effective for skin vitality, skin improvement and radiance, and a wavelength of 620 nm to 700 nm is effective for improving wrinkles and skin regeneration via promoting collagen production. In addition, a wavelength of 750 nm to 1000 nm exerts the effect of amplifying the therapeutic effect by immersing into the deep layers of the skin.

As such, light therapy is based on the principle where light energy is changed into chemical, kinetic, or thermal energy for physiochemical treatment purposes within the cells, and hence, it is crucial that light is absorbed by elements or molecules within the cells. Furthermore, it is important that the light of a specific wavelength is efficiently absorbed by a specific chromophore in the cells.

For example, the red-based wavelength is used to activate the sebaceous glands in the deep layers of the skin. Furthermore, the blue-based wavelength may be used to regulate the surface conditions of the skin by activating the keratin in the epidermis by using the photodynamic therapy (PTD).

Meanwhile, most of the LED light emitting elements are used as the light source which outputs a prescribed light for the light therapy.

As an example of a previous technology which uses an LED light emitting element as a light source, a patch-type light therapy product of “patch-type skin treatment device (Registered Korean Patent No. 10-1829984)” has been commenced. Furthermore, the previous technology above requires multiple LED chips that irradiate light of a certain wavelength range on the skin when power is supplied. In addition, it comprises an LED patch unit having a zigzag pattern in order that the circuit pattern connecting the LED chip above is flexibly changed according to the changes of the form above.

Also, as another previous technology, a “photo mask device for skin beauty (Registered Korean Patent No. 10-1074882)” has been commenced. Furthermore, this requires a light emission unit which is mounted in the face mask to emit light onto the face of a user wearing the face mask, a light emission driver unit which drives the light emission unit on/off, and an operation unit which selects the driving of the light emission unit, and a control unit which controls the light emission driving unit according to the operation signals of the operation unit.

As in the above, the devices which exert light therapy effects by using the LED light emitting elements have limitations in applicable products and production costs are quite high due to the indispensable requirement of the electric circuits for driving the LED light emitting elements and the means of power supply.

Furthermore, there is an issue in the fact that it is inconvenient to use, ineffective in terms of portability, inconvenient due to standby for charging, and the risk of exposure to electromagnetic waves as devices used for the human body.

Meanwhile, in the case of a conventional spontaneous emission axial light fluorescent substance which absorbs ambient light and which is used as the principle of axial light, the light emission time is short and hence unsatisfactory since the blue light emission region is a high energy band, and the durability is weak.

Hence, a means to replace the LED light emitting element as a light source for the light therapy is called for.

### SUMMARY

This invention has been devised to address the issue described in the above. The purpose of this invention is the spontaneous emission principle under which the LED light emitting element is to be replaced as a light source for the light therapy, while driving on its own by using ambient light as an energy source as an eco-friendly energy source without an artificial electrical energy source. This may provide a spontaneous emission type photo conversion substance for light therapy which may easily add light therapy effects to various products by outputting the wavelength required for a long time.

Another purpose of this invention is that it may be attached to various parts of the body. Furthermore, the functional patch which maximizes the light therapy effect or the treatment effect of drugs may be provided by exerting the light therapy function using ambient light without the LEDs, incandescent lamps, and other light source devices which operate with electric energy comprising the spontaneous emission type photo conversion substance for light therapy.

Another purpose of this invention that it is formed in the shape of a sheet which may be attached to the face. Furthermore, the functional mask pack which relatively maximizes the effect of mask pack may be provided by exerting the light therapy function using ambient light without the LEDs, incandescent lamps, and other light source devices which operate with electric energy comprising the spontaneous emission type photo conversion substance for light therapy.

As for this invention, in order to achieve the above purposes, M above of the chemical formula of Ma Al₂O₃ mixes raw materials consisted of inorganic oxide in line with the formula equivalence ratio of the above with at least 1 type of metal of Ba, Sr, Ca, Mg, Eu, or Dy. The resulting mixture is formed by a blue light exciting core made of a sapphire-based blue axial light fluorescent substance which is formed to output a prescribed wavelength according to the function for light therapy by delaying the light emission time with surface defect treatment conducted via burning and grinding, the photo conversion raw materials which mixed at least one among a green conversion substance, a yellow conversion substance, a red conversion substance, and a near infrared ray conversion substance, and the photo conversion raw material mixture produced by mixing them with alcohol and silicon nitride balls in the ratio of 1:3:2. Furthermore, the spontaneous emission type photo conversion substance for light therapy may be provided, comprising a photo conversion shell enveloping the blue light exciting core above.

At which time, the blue light exciting core takes on the characteristics of the range according to the formula such as 0≤a<1.

Furthermore, in the case of the inorganic oxide raw materials above, Europium or Dysprosium is added, with Al₂O₃ as the matrix corresponding to the aluminate, which is sapphire. Alternatively, it takes on the characteristic such that it is a sapphire-based blue axial light fluorescent substance achieved by adding calcium or strontium or barium or magnesium.

Together with which, the green conversion substance and the yellow conversion substance above takes on the characteristic of any of YAG (Y₃Al₅O₁₂:Ce), LuAG (Lu₃Al₅O₁₂:Ce), M=(Ca,Sr,Ba), M₂SiO₄:Eu, M₃SiO₅:Eu, MSi₂O₂N₂:Eu, α-SiAlON, or β-SiAlON.

Furthermore, the red conversion substance above takes on the characteristic of any of M=(Ca,Sr,Ba), MAlSiN₃:Eu, M₂Si₅N₈, or K₂SiF₆:Mn.

In addition, the near infrared ray conversion substance above takes on the characteristic that zinc, gallium, and scandium oxides and manganese or chromium or neodymium is included as the transition element.

Meanwhile, in order to achieve the purposes above, this invention is attached to various parts of the user's body since an adhesive substance is applied in the rear, and is consisted of the patch main unit of various sizes and shapes, and the spontaneous emission type photo conversion substance for light therapy according to any of Claims 1 through 6. Furthermore, the functional patch comprising the spontaneous emission type photo conversion substance for light therapy which comprises a means of light emission formed in the printing method may be provided in the rear which is in contact with the user's body, which is inside of the patch main unit above.

At which time, it is formed of the same material as with the patch main unit, and is also formed relatively wider than the patch main unit. One side takes on the characteristic such that it further comprises an adhesive fused fabric which is attached to the body while covering the patch main unit attached to the user's body and maintains the state of attachment of the patch main unit as an adhesive substance is applied.

Furthermore, the means of light emission above takes on the characteristic in that a grid pattern is printed across the entire rear of the patch main unit.

Furthermore, the grid pattern above takes on the characteristic such that it is made of an interval of 2 mm to 5 mm.

Furthermore, the patch main unit takes on the characteristic of comprising a drug component.

Meanwhile, as for this invention, in order to achieve the purposes above, a tonic is immersed. Furthermore, it is consisted of a mask sheet attached to the user's face and the spontaneous emission type photo conversion substance for light therapy according to any of Claims 1 through 6. A functional mask pack comprising the spontaneous emission type photo conversion substance for light therapy which comprises a means of light emission formed by a printing method in the rear which is in contact with the user's face, which is inside of the mask sheet above, may be provided.

At which time, the means of light emission takes on the characteristic where it is formed by printing in a patterned form designed across the entire rear surface of the mask sheet.

Desirably, however, the means of light emission takes on the characteristic where it is formed by printing in a grid pattern across the entire rear surface of the mask sheet.

Furthermore, the grid pattern above takes on the characteristic where it is made of an interval of 10 mm to 5 mm.

In addition, in the case of the mask sheet above, it is a functional mask consisted of the spontaneous emission type photo conversion substance for light therapy, characterized for further comprising an extension unit having a length and a width which is connected to the portion covering the forehead portion of the face and which covers the head.

This invention has the effect of replacing the LED light emitting element as a light source for light therapy according to this invention through a means of solving the above problems. Furthermore, it is a spontaneous emission principle which operates on its own by using ambient light as an energy source without an electric energy source, and also has an effect which may be applied to various light therapies by outputting the required wavelength for a very long time.

Furthermore, the functional patch according to this invention may be attached to various parts of the body, and by comprising the spontaneous emission type photo conversion substance for light therapy, it uses ambient light to convert it into a wavelength with the light therapy effect and output. Hence, it has the effect of conveniently receiving various light therapies such as pain relief, sterilization, wound infection's prevention, and the vitamin D production. Furthermore, there is an effect of maximizing the therapeutic effect of the drug depending on the added drug.

In addition, the functional mask pack according to this invention is formed in the shape of a sheet attachable to the face, and by comprising the spontaneous emission type photo conversion substance for light therapy, it executes the light therapy function using ambient light without the LEDs, incandescent lamps, and other light source devices which operate with the electric energy. Furthermore, through this, there is an effect of relatively maximizing the effect of the mask pack.

### BRIEF DESCRIPTION OF THE DRAWINGS

FIG. 1 is a drawing intended for conceptually describing the spontaneous emission type photo conversion substance for light therapy according to this invention.

FIG. 2 is a flow chart illustrating a method of preparing a spontaneous emission type photo conversion substance for light therapy according to this invention.

FIG. 3A is an SEM photo before the surface treatment phase of the sapphire-based blue axial light fluorescent substance contained in the spontaneous emission type photo conversion substance for light therapy according to this invention. FIG. 3b is an SEM photo after the surface treatment phase of the sapphire-based blue axial light fluorescent substance contained in the spontaneous emission type photo conversion substance for light therapy according to this invention.

FIG. 4A is the light emission spectrum and FIG. 4B is the light emission time spectrum of the sapphire-based blue axial light fluorescent substance included in the spontaneous emission type photo conversion substance for light therapy according to this invention.

FIGS. 5A-5B are SEM photos of the photo conversion shell contained in the spontaneous emission type photo conversion substance for light therapy according to this invention.

FIGS. 6A-6B are photos of the spontaneous emission type photo conversion substance for light therapy according to this invention.

FIG. 7 is a photo of comparison of the spontaneous emission according to the formation state of the photo conversion shell contained in the spontaneous emission type photo conversion substance for light therapy according to this invention.

FIG. 8 is a photo of comparison of the spontaneous emission according to the content ratio of the sapphire-based blue axial light fluorescent substance and the photo conversion substance contained in the spontaneous emission type photo conversion substance for light therapy according to this invention.

FIG. 9 is a photo of comparison of the spontaneous emission according to the content ratio of the red/green/blue conversion substance of the spontaneous emission type photo conversion substance for light therapy according to this invention.

FIG. 10 is a drawing illustrating an example of use and the form of the functional patch containing the spontaneous emission type photo conversion substance for light therapy according to this invention.

FIG. 11 is a drawing intended for describing the conceptual features of the functional patch containing the spontaneous emission type photo conversion substance for light therapy according to this invention.

FIG. 12 is a drawing intended for describing the means of light emission included in the functional patch containing the spontaneous emission type photo conversion substance for light therapy of this invention.

FIG. 13 is a schematic drawing of the functional mask pack comprising the spontaneous emission type photo conversion substance for light therapy according to this invention.

FIG. 14 is a drawing intended for describing the conceptual features of a functional mask pack comprising the spontaneous emission type photo conversion substance for light therapy according to this invention.

FIG. 15 is a drawing illustrating another implementation example of the mask sheet illustrated in Drawings 13 and 14.

FIGS. 16A-16D are drawings intended for describing the means of light emission included in the functional mask pack comprising the spontaneous emission type photo conversion substance for light therapy of this invention.

FIG. 17 is a light spectrum of the preparing example 1 according to the preparing method of the spontaneous emission type photo conversion substance for light therapy according to this invention.

FIG. 18 is a light spectrum of the preparing example 2 according to the preparing method of the spontaneous emission type photo conversion substance for light therapy according to this invention.

FIG. 19 is a light spectrum of the preparing example 3 according to the preparing method of the spontaneous emission type photo conversion substance for light therapy according to this invention.

FIG. 20 is a light spectrum of the preparing example 4 according to the preparing method of the spontaneous emission type photo conversion substance for light therapy according to this invention.

FIG. 21 is a light spectrum of the preparing example 5 according to the preparing method of the spontaneous emission type photo conversion substance for light therapy according to this invention.

FIG. 22 is a light spectrum of the preparing example 6 according to the preparing method of the spontaneous emission type photo conversion substance for light therapy according to this invention.

FIG. 23 is a light spectrum of the preparing example 7 according to the preparing method of the spontaneous emission type photo conversion substance for light therapy according to this invention.

FIG. 24 is a light spectrum of the preparing example 8 according to the preparing method of the spontaneous emission type photo conversion substance for light therapy according to this invention.

FIGS. 25A-25B illustrates a sterilization power comparison testing drawing of the implementation example 1 where a means of light emission consisted of the spontaneous emission type photo conversion substance for light therapy according to this invention is formed.

FIG. 26 is a graph of the results of the skin density test of the implementation example 1 where a means of light emission consisted of the spontaneous emission type photo conversion substance for light therapy according to this invention is formed.

FIG. 27 is a graph of the results of the pigmentation experiment of the implementation example 1 where a means of light emission consisted of the spontaneous emission type photo conversion substance for light therapy according to this invention is formed.

### DESCRIPTION OF THE REFERENCE NUMERALS

*6410: Spontaneous emission type photo conversion substance for light therapy
12: blue light exciting core
*6614: photo conversion shell
100: functional patch
101: patch main unit
110: means of light emission
200: functional mask pack
201: mask sheet
210: means of light emission

### DETAILED DESCRIPTION OF THE EMBODIMENTS

Advantages and special characteristics of this invention, and a method of achieving them will become apparent by making reference to the preparing examples described below in detail along with the attached drawings. However, this invention is not limited to the preparing examples disclosed below, but rather will be implemented across various different forms. Furthermore, these preparing examples are provided merely to complete the disclosure of this invention, and to completely inform a person of conventional skills in the technical area in which this invention belongs. In addition, this invention is only defined by the scope of the claims.

By making reference to the attached drawings, the specifics for the implementation of this invention will be described in detail. Regardless of the drawings, the same reference numbers refer to the same components, and “and/or” comprises each and all combinations of one or more of the mentioned items.

The terms used hereunder are intended for describing the preparing examples and are not intended to limit this invention. In this specification, the singular form also comprises the plural form unless specifically provided in the text. Used in the specification, “comprises” and/or “comprising” do not exclude the existence or addition of one or more other components other than the mentioned components.

Unless otherwise defined, any and all terms (comprising technical and scientific terms) used hereunder may be used in the technical area where this invention pertains to those of ordinary skills to which this invention belongs. Furthermore, the terms defined in a commonly used dictionary are not interpreted ideally or excessively unless explicitly defined specifically.

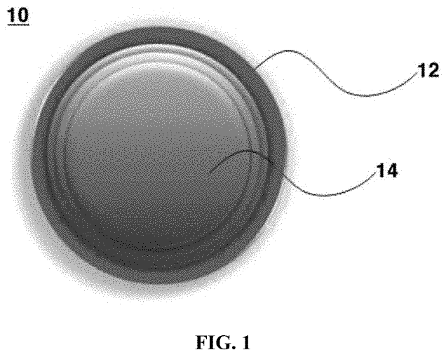

The spontaneous emission type photo conversion substance for light therapy (10) according to this invention is applied to various products and may add the light therapy effect without electrical energy. This will describe in detail the spontaneous emission type photo conversion substance for light therapy and the preparing method according to this invention by making reference to the attached FIGS. 1 to 9 below.

FIG. 1 is a drawing intended for conceptually describing the spontaneous emission type photo conversion substance for light therapy according to this invention.

As illustrated in FIG. 1, the spontaneous emission type photo conversion substance for light therapy (10) has a composite structure consisting of a blue light exciting core (12) positioned at the center and the photo conversion shell (14) surrounding the blue light exciting core (12).

At which time, the blue light exciting core (12) is made of a sapphire-based blue axial light fluorescent substance, and the photo conversion shell above (14) is made of a photo conversion substance. Thus, in the case of the spontaneous emission type photo conversion substance for light therapy (10), it has a relatively long light emission time and absorbs sunlight and ambient light, absorbs the exciting light above in the photo conversion shell above (14) enveloping the blue light exciting core above (12) which outputs the exciting light, thereby executing the photo conversion with the wavelength selected for light emission as a matter of technical characteristic.

That is, in the case of the spontaneous emission type photo conversion substance for light therapy (10), the exciting light output from the blue light exciting core above (12) is absorbed by the photo conversion shell (14) and converted into a prescribed wavelength for light therapy, after which it outputs as the light of the wavelength having the light therapy effect, and hence, is a fluorescent substance which executes spontaneous emission.

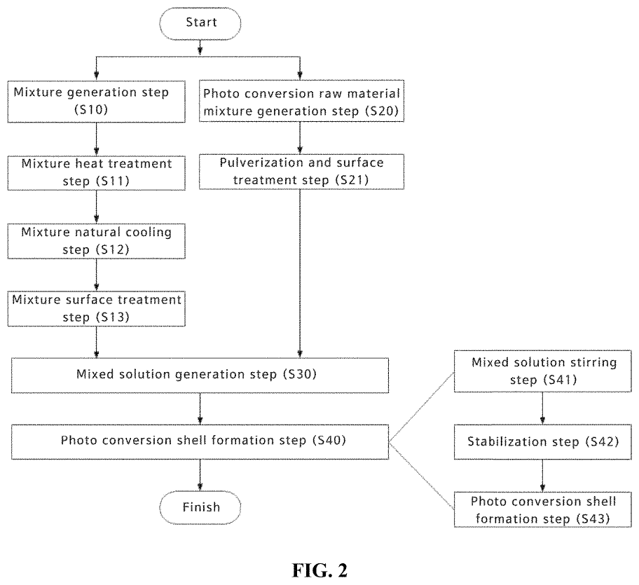

FIG. 2 is a flow chart illustrating a method of preparing the spontaneous emission type photo conversion substance for light therapy according to this invention.

As illustrated in FIG. 2, in preparing the spontaneous emission type photo conversion substance for light therapy (10) consisting of the blue light exciting core above (12) and the photo conversion shell above (14) enveloping the blue light exciting core (12), the sapphire-based blue axial light fluorescent substance forming the blue light exciting core (12) undergoes largely a mixture generation step (S10), mixture heat treatment step (S11), mixture natural cooling step (S12), and mixture surface treatment step (S13). Furthermore, the photo conversion substance forming the photo conversion shell above (14) is prepared in the form of a slurry comprising the photo conversion raw material mixture generation step (S20) and the grinding and surface treatment step (S21). In addition, the spontaneous emission type photo conversion substance for light therapy (10) is prepared through the mixed solution generation step (S30) and the photo conversion shell formation step (S40) by using the prepared sapphire blue axial light fluorescent substance and the photo conversion substance in a slurry form.

Specifically, in order to prepare the sapphire-based blue axial light fluorescent substance forming the blue light exciting core above (12) of the spontaneous emission type photo conversion substance for light therapy (10), the mixture generation step above (S10) produces the mixture by mixing raw materials made of inorganic oxides in line with the above chemical equivalence ratio with metals of 1 type or more of Ba, Sr, Ca, Mg, Eu, or Dy for M above of the chemical formula of Ma Al₂O₃.

At which time, the range according to the formula of the mixture generation step above (S10) is desirable to be 0≤a<1. Furthermore, the inorganic oxide raw materials may add Europium or Dysprosium for matrix as for Al₂O₃ which corresponds to aluminate, which is sapphire, or add Calcium or Strontium or Barium or Magnesium for formation.

In the heat treatment of the mixture above (S11), the mixture generated in the heat treatment of the mixture above (S10) is heated to perform the heat treatment.

In which event, the heat treatment of the mixture step above (S11) may be performed by heating at 1,000° C. to 1,500° C. for 2 to 12 hours, and the heat treatment of the mixture as such may be performed in a reducing atmosphere.

In the natural cooling of the mixture above (S12), the mixture which is heat treated through the heat treatment of the mixture above (S11) is gradually and naturally cooled at the room temperature.

In the mixture surface treatment step above (S13), the mixture naturally cooled through the mixture natural cooling step above (S12), and the alcohol and silicon nitride balls are mixed at the ratio of 2:1:2. Following which, the mixture is stirred at 1,000 rpm to 5,000 rpm for 1 hour to 5 hours, and the mixture above is crushed with physical force to treat the surface defects.

In the case of the mixture surface treatment step above (S13), the spontaneous emission type photo conversion substance for light therapy above (10) is performed in order to have a relatively longer afterglow characteristic. At which time, it is desirable to control the particle size of the crushed mixture to be formed in the range of 15 μm to 20 μm.

Meanwhile, in order to prepare the photo conversion substance forming the photo conversion shell (14), first, the photo conversion raw material mixture generation step above (S20) produces the photo conversion raw material mixture by mixing the photo conversion raw material and alcohol and silicon nitride ball which mix any one of or more than one of the substances belonging to the green conversion substance, which is identical to the photo conversion raw material listed in Table 1, yellow conversion substance, red conversion substance, and the near infrared ray conversion substance at the ratio of 1:3:2.

TABLE 1

| | | Absorption | Photo |
|---|---|---|---|
| | | wavelength | conversion |
| Substance | Mixture | band | area |
| Aluminate | Y₃Al₅O₁₂:Ce³⁺ | UV~Green | Green~yellow |
| | Lu₃Al₅O₁₂:Ce³⁺ | | |
| | Tb₃Al₅O₁₂:Ce³⁺ | | |
| Silicate | M₂SiO₄:Eu²⁺(M═Ca,Sr,Ba) | | Green~yellow |
| | M₂SiO₄:Eu²⁺ | | |
| | K₂SiF₆:Mn⁴⁺ | | Red~near |
| | | | infrared ray |
| Oxy-nitride | β-SiAlON | | Green~yellow |
| | α-SiAlON | | |
| | MSi₂O₂N₂:Eu²⁺(M═Sr,Ba) | | |
| Nitride | MAlSiN₃:Eu²⁺(M═Ca,Sr) | | Red~near |
| | M₂Si₅N₈:Eu(M═Ca, Sr,Ba) | | infrared ray |
| Others | Zn₃Ga₂Ge₂O₁₂ | | Near infrared |
| | Zn₂GaO₄ | | ray~far |
| | | | infrared |

That is, in the case of the photo conversion raw material forming the photo conversion shell above (14), as provided in Table 1, any one of or more than one of the green conversion substance, yellow conversion substance, red conversion substance, and the near infrared ray conversion substance can be formed in combination. Furthermore, the green conversion substance and the yellow conversion substance may be YAG (Y₃Al₅₀₁₂:Ce), LuAG (Lu₃Al₅O₁₂:Ce), M=(Ca,Sr,Ba), M₂SiO₄:Eu, M₃SiO₅:Eu, MSi₂O₂N₂:Eu, α-SiAlON, β-SiAlON, etc. In addition, the red conversion substance above may be, as an example of the red conversion substance as such, M=(Ca,Sr,Ba), MAlSiN₃:Eu, M₂Si₅N₈, K₂SiF₆:Mn, etc.

*94 Furthermore, the near infrared ray conversion substance above comprises zinc, gallium, and scandium oxides and manganese or chromium or neodymium as transition elements.

The crushing and surface treatment step above (S21) is a process intended for producing a slurry form by crushing and surface treating the photo conversion raw material mixture which is generated in the photo conversion raw material mixture generating step above (S20) by using a planetary ball mill method. At which time, it is desirable that the planetary ball mill be 5,000 rpm or more.

The photo conversion substance prepared as such forms the photo conversion shell above (14) outputs the proven wavelength for pain relief, sterilization, prevention of wound infection, and the vitamin D formation by taking the input of the wavelength of the ultraviolet to blue area output from the sapphire-based blue axial light fluorescent substance forming the blue light exciting core above (12).

For example, the red conversion substance above or the near infrared ray conversion substance outputs light of the wavelength which aids skin regeneration.

That is, the spontaneous emission type photo conversion substance for light therapy above (10) is formed from the sapphire-based blue axial light fluorescent substance and the photo conversion substance. Hence, while outputting the wavelength of the blue area of 400 nm to 500 nm, which exerts effects such as pain relief, sterilization, wound infection prevention, and vitamin D generation, it also outputs the near ultraviolet rays effective for atopy, or 780 nm, which are effective for skin regeneration, or can output the near infrared ray of 2.5 μm wavelength.

Meanwhile, in the case of the mixture solution generation step above (S30) to form the complex structure of the spontaneous emission type photo conversion substance for light therapy (10), a mixed solution is produced by mixing the sapphire-based blue axial light fluorescent substance prepared through the mixture generation step above (S10) or the photo conversion raw material mixture generation step (S20) and the photo conversion substance above in the form of a slurry prepared through the photo conversion raw material mixture generation step above (S20) or the crushing and surface treatment step above (S21).

Thereafter, through the photo conversion shell formation step above (S40), the mixed solution above generated in the mixed solution generation step (S30) is heat treated whereby the photo conversion shell above (14) made of the photo conversion substance above will be formed at the blue light exciting core above (12) made of the sapphire blue axial light fluorescent substance.

Specifically, in the case of the photo conversion shell formation step above (40), sodium oleate and dispersant for attaching the sapphire-based blue axial light fluorescent substance above and the photo conversion substance above to the mixed solution generated in the mixed solution generation step above (S30) will be added. After which, alcohol is removed by heating the mixed solution above stabilized through the stabilization step (S42) and the stabilization step above (S42) of letting the stirred mixed solution to be left at the room temperature for 1 to 3 hours after undergoing the mixed solution stirring step (S41) and the mixed solution stirring step (S41) for 30 to 120 minutes. In addition, this comprises a photo conversion shell formation step (S43) of forming a photo conversion shell made of a photo conversion substance on the blue light exciting core.

At which time, it is desirable that the stabilization step above (S42) further comprises the washing step of washing by using ethanol or ultrapure water.

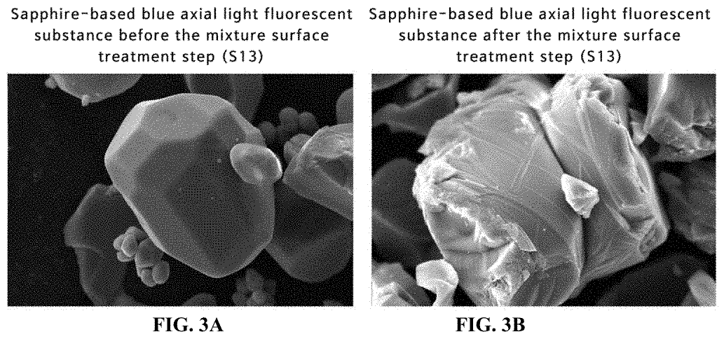

FIG. 3A is an SEM photo before the surface treatment step of the sapphire-based blue axial light fluorescent substance included in the spontaneous emission type photo conversion substance for light therapy according to this invention. FIG. 3B is an SEM photo after the surface treatment step of the sapphire-based blue axial light fluorescent substance included in the spontaneous emission type photo conversion substance for light therapy according to this invention.

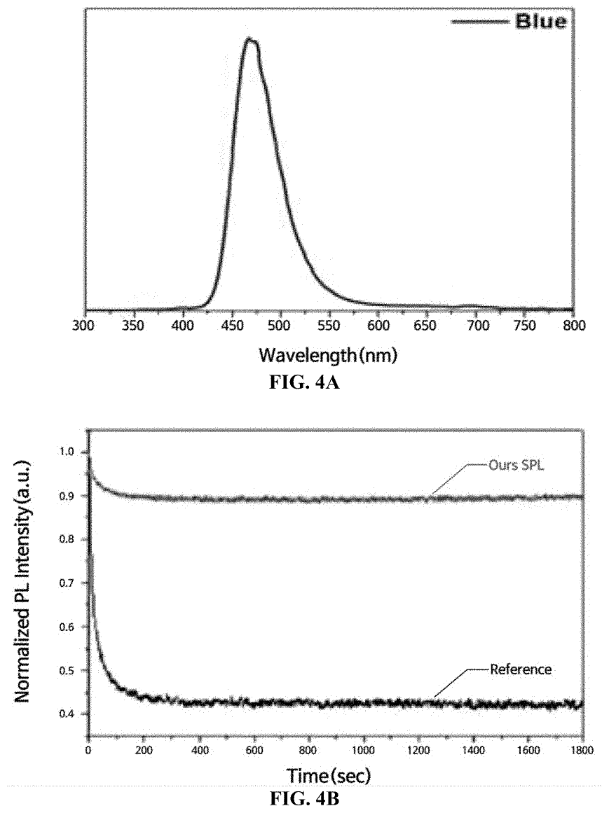

In addition, FIG. 4A is the light emission spectrum and FIG. 4B is the light emission time spectrum of the sapphire-based blue axial light fluorescent substance included in the spontaneous emission type photo conversion substance for light therapy according to this invention.

As compared through FIGS. 3A-3B, in the method of preparing the spontaneous emission type photo conversion substance for light therapy above (10), in the mixture surface treatment step above (S13), the surface defect treatment of the sapphire-based blue axial light fluorescent substance is performed.

This is intended to ensure that the spontaneous emission type photo conversion substance for light therapy above (10) has a relatively longer long afterglow characteristic, and it can be known that as for such characteristic, the light emission time is longer than before, as illustrated FIG. 4B.

In particular, the sapphire-based blue axial light fluorescent substance above absorbs ambient light by controlling the particle size of 15 to 20 μm by physical force and performing surface defect treatment through the mixture surface treatment step above (S13). Hence, as illustrated FIG. 4A, it can be verified that the wavelength of 400 to 550 nm is light emitted and output for 30 minutes or longer.

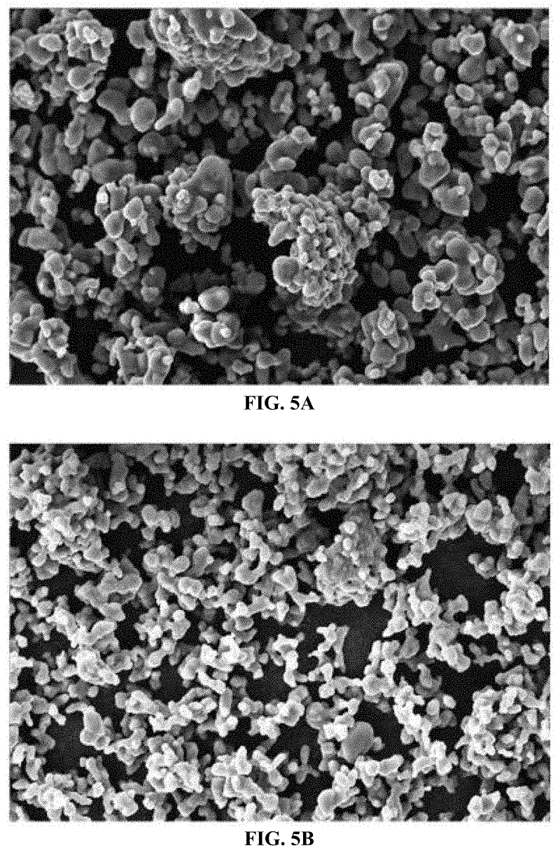

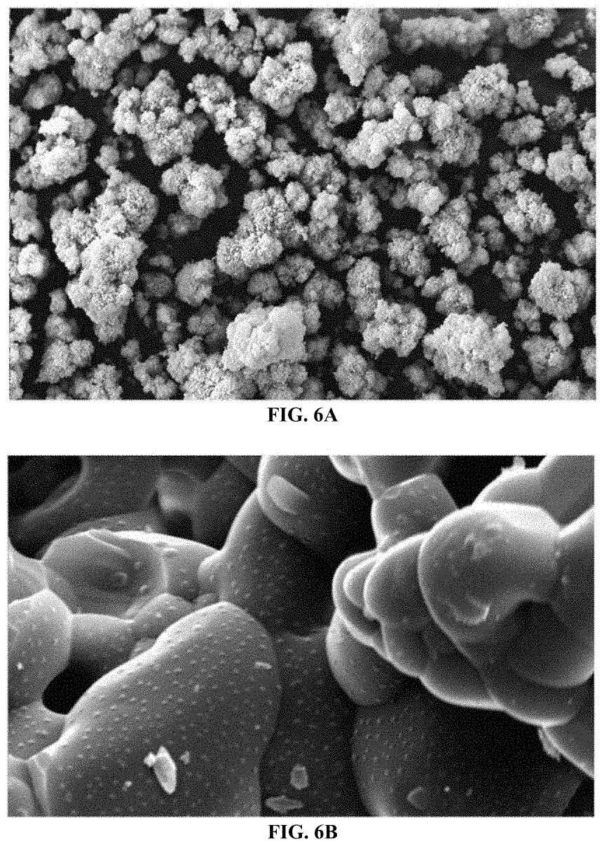

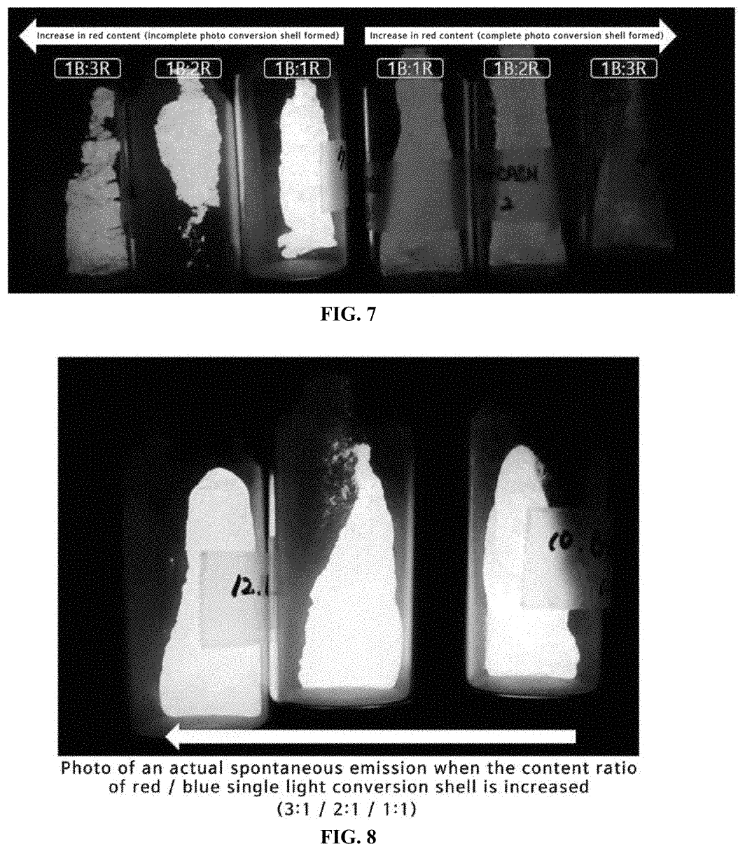

FIGS. 5A-4B are SEM photos of a photo conversion shell contained in the spontaneous emission type photo conversion substance for light therapy according to this invention. Furthermore, FIGS. 6A-6B are photos of the spontaneous emission type photo conversion substance for light therapy according to this invention. In addition, FIG. 7 is a photo of the comparison of the spontaneous emission according to the formation state of the photo conversion shell included in the spontaneous emission type photo conversion substance for light therapy according to this invention.

As examined earlier, through the mixed solution generating step above (S30), the sapphire-based blue axial light fluorescent substance illustrated in FIGS. 4A-4B and the photo conversion substance illustrated in FIGS. 5A-5B are mixed to generate a mixed solution. Furthermore, this forms the photo conversion shell above (14) made of the photo conversion substance on the blue light exciting core above (12) made of the sapphire-based blue axial light fluorescent substance through the photo conversion shell formation step above (S40).

At which time, the photo conversion shell above (14) formed outside of the blue light exciting core above (12) in the photo conversion shell formation step (S40) is formed differently depending on the temperature and time. Furthermore, the higher the temperature and the longer it is heated, the photo conversion shell is formed while completely enveloping the blue light exciting core, as illustrated in FIG. 6A. Furthermore, when heating is performed at a relatively low temperature or for a short time, a photo conversion shell is formed incompletely as illustrated in FIG. 6B.

Furthermore, as verified through FIG. 7, when looking at the photo of the comparison of the spontaneous emission of the spontaneous emission type photo conversion substance for light therapy above (10) formed by mixing the sapphire-based blue axial light fluorescent substance (B) and the red conversion substance (R) at the ratio of 1:1 to 1:3, it can be seen that the blue light emission converts inefficiently in photo conversion, when an incomplete photo conversion shell if formed. In addition, when a complete photo conversion shell is formed, it can be seen that the efficiency of the photo conversion is high since the structure is one of overall enclosure.

Hence, since the photo conversion shell above (14) is completely or incompletely formed on the blue light exciting core above (12), the spontaneous emission wavelengths is generated differently from each other. In addition, when the spontaneous emission type photo conversion substance for light therapy above (10) is completely formed by being enveloped entirely by the photo conversion shell above (14) on the blue light exciting core above (12), the efficiency is high. Whereas, when the photo conversion shell above (14) is incompletely formed, the photo conversion substance is sparsely attached to the blue light exciting core, and hence, the conversion efficiency will decline.

However, when selecting the wavelength of light therapy, if it is necessary to select functions such as sterilization and disinfection, which are blue functions, with priority, it is desirable that the photo conversion shell above (14) use the spontaneous emission type photo conversion substance for light therapy above (10) which is incompletely formed as in FIG. 6B.

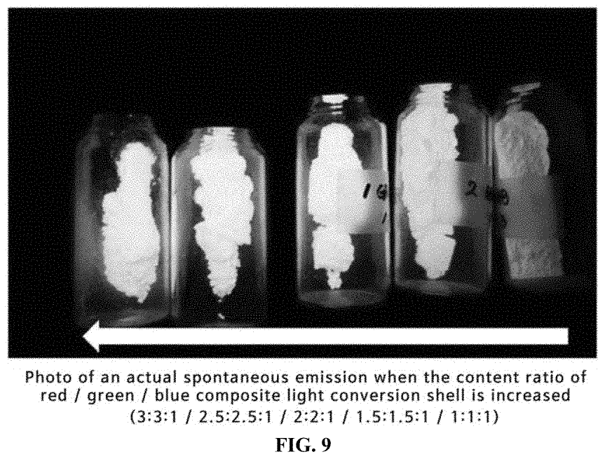

FIG. 8 is a photo of the comparison of the spontaneous emission according to the content ratio of the sapphire-based blue axial light fluorescent substance and the photo conversion substance contained in the spontaneous emission type photo conversion substance for light therapy according to this invention. FIG. 9 is a photo of the comparison of the spontaneous emission according to the content ratio of red/green/blue conversion substance of the spontaneous emission type photo conversion substance for light therapy according to this invention.

As illustrated in FIG. 8, it can be seen that, as the content ratio of the photo conversion substance forming the photo conversion cell to the sapphire-based blue axial light fluorescent substance is increased, the light emission color of the photo conversion substance forming the photo conversion cell is increased. Furthermore, as illustrated in FIG. 9, it is possible to implement various light emission colors by controlling the content ratio while using the photo conversion substance forming the photo conversion shell in combination.

Hence, the light emission color can be adjusted by regulating the content ratio of the photo conversion substance above to the sapphire-based blue axial light fluorescent substance above.

Meanwhile, the spontaneous emission type photo conversion substance for light therapy according to this invention can be applied as a means of light emission for various products to add light therapy effect without electrical energy. In particular, it can be applied as a means of light emission such as a skin cosmetic or medical patch or a skin cosmetic mask pack.

Hereinbelow, by making reference to the attached Drawings 10 through 12, as for the form of implementation of this invention, a functional patch comprising a spontaneous emission type photo conversion substance for light therapy will be described.

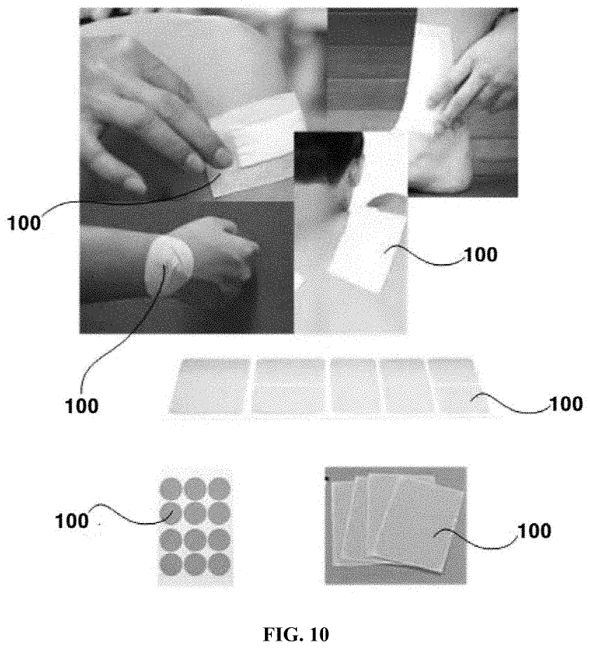

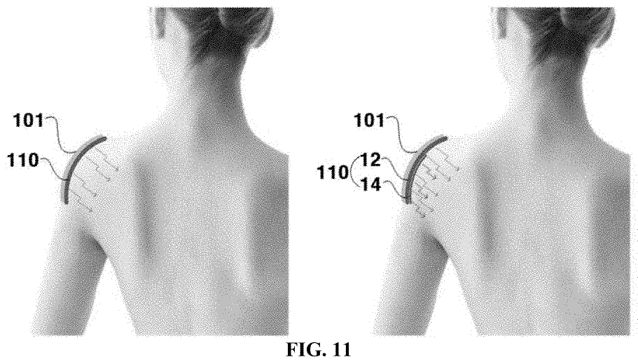

FIG. 10 is a drawing illustrating an example of use and the form of a functional patch containing a spontaneous emission type photo conversion substance for light therapy according to this invention. Furthermore, FIG. 11 is a drawing intended for describing the conceptual features of the functional patch comprising a spontaneous emission type photo conversion substance for light therapy according to this invention.

As illustrated in FIG. 10 or FIG. 11, the functional patch (100) comprising the spontaneous emission type photo conversion substance for light therapy of this invention is formed in the printing method by which the means of light emission (11) formed by the spontaneous emission type photo conversion substance for light therapy above (10) in the rear of the patch main unit (101) whose materials are basically same as the general path traditionally used. In addition, the method of use is identical to that of the general patch.

At which time, the patch main unit above (101) can be formed in various shapes and sizes such as a square, a rectangle, a circle, and an oval so that it can be attached to and used on various parts of the body. In addition, it is made of a fibrous material, yet has a thickness of several mm and has an overall elasticity so that it can be easily adhered to a curved part of the skin.

Desirably, as an implementation example of the patch main unit above (101), it can be made of a fibrous material, and in particular, it is desirable that it be formed of a printable sheet of fibrous material such as a printable cotton sheet, non-woven sheet, and cellulose sheet, etc. Furthermore, more desirably, it can be made of TENCEL fabric.

As it is known, TENCEL fabric is a functional natural material developed by Lenzing, an Austrian textile company, and is also an eco-friendly material made of eucalyptus tree extracts, and hence, has excellent moisture contents and water absorption. Furthermore, it is effective for sensitive skin as the fiber structure is smooth and it does not irritate the skin.

Furthermore, as another implementation example of the patch main unit above (101), it can be made of a hydrogel, a material having a three-dimensional hydrophilic polymer network structure using purified water as a dispersion medium in order to improve adhesion to the skin and moisturizing ability. At which time, the hydrogel forming the patch main unit above (101) can be formed of various viscoelastic polymers known in the industry such as hyaluronic acid, agarose, alginate, chitosan, gelatin or collagen.

Furthermore, it is apparent that the patch main unit above (101) is not limited to the aforesaid implementation examples and can also be implemented in other forms.

In addition, an adhesive material is applied in the rear of the patch main unit above (101), that is, the inner surface of the patch main unit above (10) which directly contacts the skin, which makes attachment to the body easy.

Moreover, it is formed relatively wider than the patch main unit above (101) and an adhesive material is applied on one side. Hence, it may further include an adhesive fabric (102) attached to the body while covering the patch main unit (101) attached to the user's body to maintain the attachment state of the patch main unit (101). The adhesive fabric above (102) may generally be a conventional adhesive fabric used to maintain the attachment state of the patch and prevent contamination from against the outside, and may also be formed of the same material as the patch main unit above (101).

Meanwhile, when the functional patch (100) comprising the spontaneous emission type photo conversion substance for light therapy of this invention is used for the prescribed therapeutic purpose, it can comprise various pharmaceutical ingredients according to the therapeutic purpose.

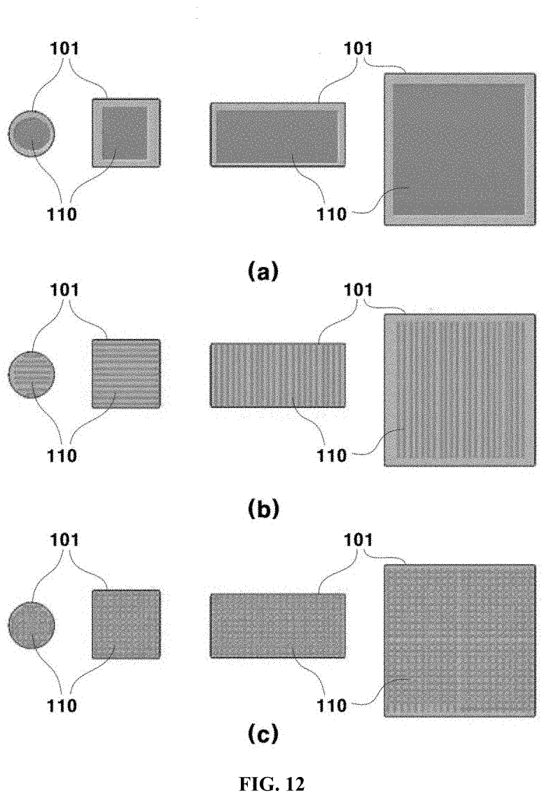

FIG. 12 is a drawing intended for describing the means of light emission included in the functional patch comprising the spontaneous emission type photo conversion substance for light therapy of this invention.

As illustrated in FIG. 12, the means of light emission above (110) is formed by printing in the rear of the patch main unit above (101), and can be formed by printing in a patterned form designed across the entire rear surface of the patch main unit above (101).

Specifically, the patch main unit above (101) can be printed in the entire rear surface of the patch main unit above (101), and can be printed in the entire rear surface of the patch main unit above (101) in a striped pattern, or can be printed in a grid pattern.

When the means of light emission above (110) is formed by printing in a stripe pattern or a grid pattern, the entire face may sufficiently be provided with the light therapy effect while reducing the raw materials required to form the means of light emission above (110) relative to the case where the means of light emission above (110) is printed across the entire rear surface.

Here, when the means of light emission above (110) is printed in the form of a stripe pattern or a grid pattern in the rear surface of the patch main unit above (101), it is desirable that the spacing between the lines and the lines or between the grids be made of a distance between 2 mm and 5 mm.

Furthermore, the means of light emission above (110) is not limited to the form illustrated in FIG. 12, and may also be printed in various shapes or patterns as necessary, and is not limited to any specific pattern or design.

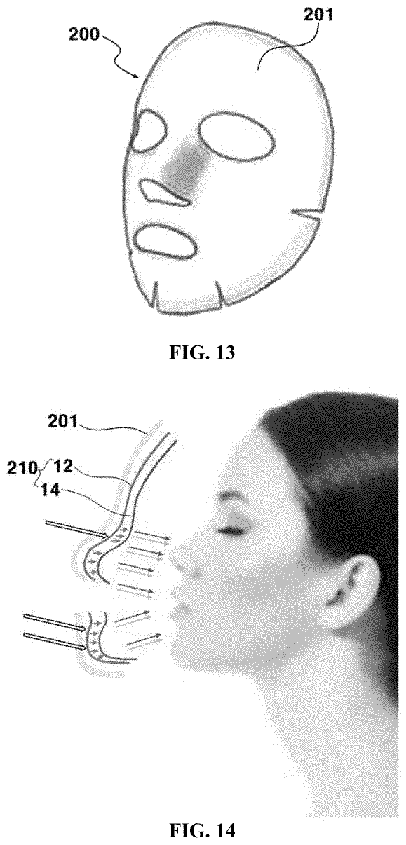

Hereinbelow, by making reference to the attached FIGS. 13 to 16, as for another implementation form of this invention, a functional patch comprising a spontaneous emission type photo conversion substance for light therapy will be described.

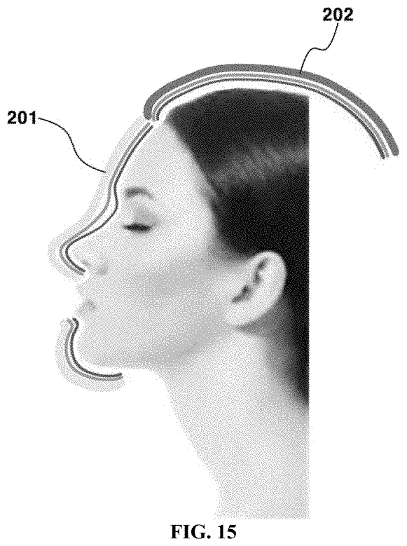

FIG. 13 is a drawing of a functional mask pack comprising the spontaneous emission type photo conversion substance for light therapy according to this invention. Furthermore, FIG. 14 is a drawing intended for describing the conceptual features of the functional mask pack comprising a spontaneous emission type photo conversion substance for light therapy according to this invention. FIG. 15 is a drawing illustrating another implementation example of the mask sheet illustrated in FIGS. 13 and 14.

As illustrated in FIG. 13 through FIG. 15, the functional mask pack (200) comprising the spontaneous emission type photo conversion substance for light therapy of this invention is formed in the printing method of the light emission method (210) executed by the spontaneous emission type photo conversion substance for light therapy above in the rear of the mask sheet of the form of a sheet in which lotion is immersed as with conventional mask packs, whose use is identical to that of general sheet type mask packs.

First, the mask sheet above (201) is formed in a shape corresponding to it, so as to be in close contact with the user's face to cover all or some part of the user's face.

Furthermore, the mask sheet above (201), as an implementation example, may be made of a fibrous material, and in particular, it is desirable that it be formed of a printable fibrous material sheet such as a printable cotton sheet, a non-woven sheet, a cellulose sheet, and more desirably, it can be made of TENCEL fabric.

As it is known, TENCEL fabric is a functional natural material developed by Lenzing, an Austrian textile company and is also an eco-friendly material made of eucalyptus tree extract. It has excellent moisture content and absorption, and its smooth fiber structure does not irritate the skin. Hence, it is effective for sensitive skin.

Furthermore, as another implementation example, the mask sheet above (201) may be made of a hydrogel, a material having a three-dimensional hydrophilic polymer network structure using purified water as a dispersion medium in order to improve adhesion and moisturizing ability with the skin. At which time, the hydrogel forming the mask sheet above (201) can be formed by comprising various viscoelastic polymers publicly known in the industry such as hyaluronic acid, agarose, alginate, chitosan, gelatin or collagen.

In addition, it is apparent that the mask sheet above (201) is not limited to the above-mentioned implementation examples and may be implemented in other forms.

Furthermore, the lotion immersed in the mask sheet above (201) can be a material of various natural or chemical components which are helpful for the skin, such as whitening, whitening, nutrition, and wrinkle improvement, depending on the component, and is not limited to a specific material.

In addition, the mask sheet above (201) may further comprise an extension unit (202) having a length and a width connected to a part covering the forehead of the face to cover the head.

The extension unit above (202) has a length and width that can cover the head, so that the functional mask pack (200) comprising the spontaneous emission type photo conversion substance for light therapy of this invention executes light therapy functions on not only the user's face, but also on the scalp of the head.

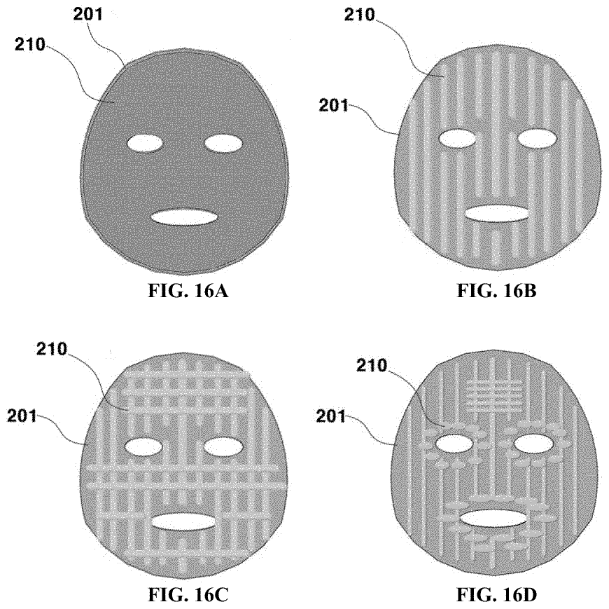

FIGS. 16A-16D are drawings intended for describing the means of light emission comprising in the functional mask pack comprising the spontaneous emission type photo conversion substance for light therapy of this invention.

As illustrated in FIGS. 16A-16D, the means of light emission above (210) is formed by printing in the rear of the mask sheet above (201), and is also formed by printing in a patterned form designed over the entire rear surface of the mask sheet above (201).

Specifically, the mask sheet above (201) may be printed across the entire rear surface of the mask sheet above (201), and may also be printed on the entire rear surface of the mask sheet above (201) in a striped pattern or printed in a grid pattern.

When the means of light emission above (210) is formed by printing in a stripe pattern or a grid pattern, the light therapy effect can be provided sufficiently across the entire face while reducing the raw materials required to form the means of light emission above (210) relative to the case where the means of light emission above (210) is printed across the entire rear surface.

Here, when the means of light emission above (210) is printed in the form of a stripe pattern or a grid pattern in the rear of the patch main unit above (201), it is desirable that the spacing between the lines and the lines or between the grids be made in 5 mm to 10 mm.

Moreover, when intensive care is required on a specific area of the face such as around the eyes, around the lips, and the forehead, the means of light emission above (210) should ensure that rear area of the mask sheet above (201) corresponding to a specific area such as around the eyes, around the lips, and forehead is printed in a relatively large area relative to other areas such as cheeks and chin, so that a relatively large amount of light is supplied to the skin.

Various forms in which the means of light emission above (210) are printed in the rear surface of the mask sheet above (10) have been described through FIGS. 16A-16D. However, the means of light emission above (210) is not limited to the illustrated forms, and may also be printed in a pattern or design in various forms as needed, and is not limited to any specific pattern or design.

Hereinbelow, this invention will be described more specifically via a preparing example, an implementation example, and an experimental example according to the method of preparing the spontaneous emission type photo conversion substance for light therapy according to this invention.

### Preparing Example 1

The spontaneous emission type photo conversion substance for light therapy prepared through the method of preparing the spontaneous emission type photo conversion substance for light therapy of this invention was prepared by mixing at the weight ratio of “1” for the sapphire blue axial light fluorescent substance to “3” for the yellow conversion substance.

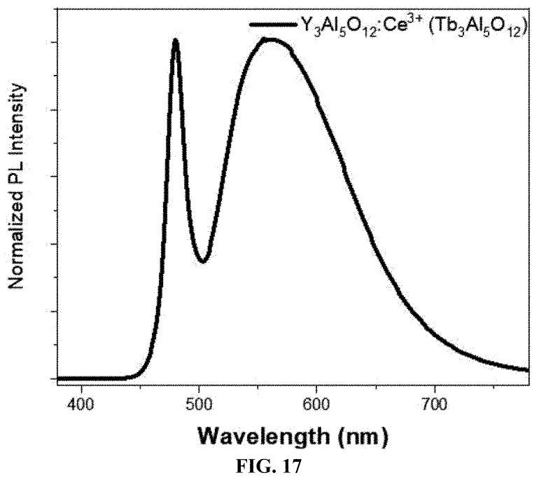

Consequently, as illustrated in the light spectrum of the preparing example 1 according to the preparing method of the spontaneous emission type photo conversion substance for light therapy according to this invention of FIG. 17, the optical properties are 480 nm for blue, 565 nm for the excitation light emission peak and yellow, where the photo conversion peak was verified in the light spectrum.

At which time, the particle size of the sapphire-based blue axial light fluorescent substance is 20.4 μm based on D50, and 2.41 μm based on D50 of Y₃Al₅O₁₂:Ce³⁺ yellow conversion substance. Furthermore, the particle size of the final spontaneous emission type photo conversion substance for light therapy was 25.3 μm based on D50.

### Preparing Example 2

The spontaneous emission type photo conversion substance for light therapy prepared through the method of preparing the Spontaneous emission type photo conversion substance for light therapy of this invention was prepared by mixing at the weight ratio “1” for the sapphire blue axial light fluorescent substance to “3” for the green conversion substance.

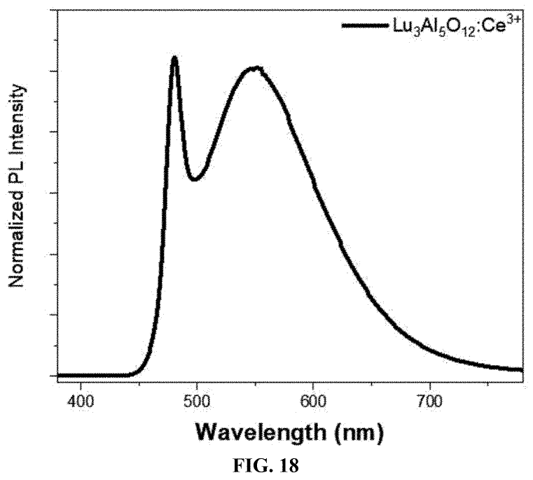

Consequently, as illustrated in the light spectrum of the preparing example 2 according to the preparing method of the spontaneous emission type photo conversion substance for light therapy according to this invention of FIG. 18, the optical properties are 480 nm blue and 540 nm for the excitation light emission peak and yellow, where the photo conversion peak was verified in the light spectrum.

At which time, the particle size of the sapphire-based blue axial light fluorescent substance is 20.4 μm based on D50 and 4.12 μm based on D50 of Lu₃Al₅O₁₂:Ce³⁺ green conversion substance. Furthermore, the particle size of the final spontaneous emission type photo conversion substance for light therapy was 21.3 μm based on D50.

### Preparing Example 3

The spontaneous emission type photo conversion substance for light therapy prepared through the method of preparing the spontaneous emission type photo conversion substance for light therapy of this invention was prepared by mixing at the weight ratio of “1” for the sapphire blue axial light fluorescent substance to “3” for the green/yellow conversion substance.

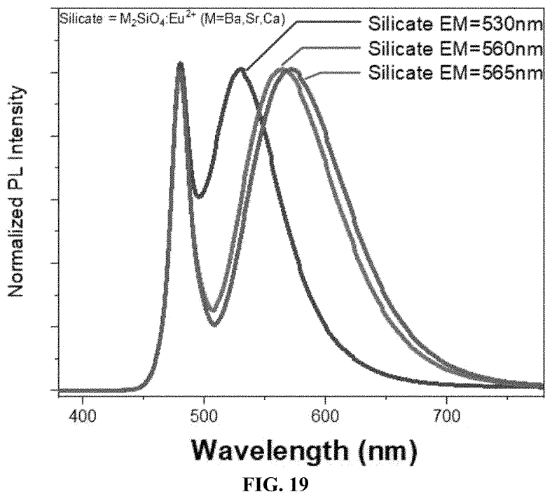

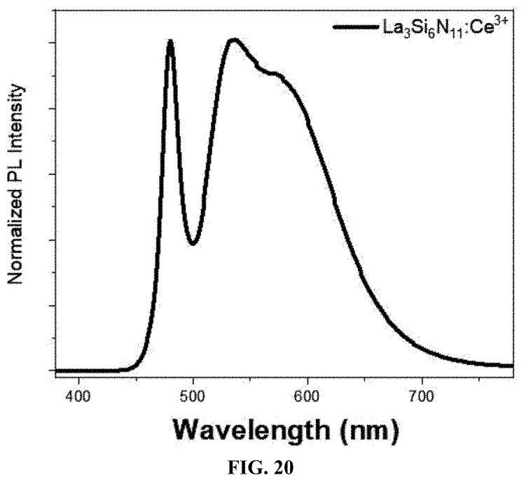

Consequently, as illustrated in the light spectrum of the preparing example 3 according to the preparing method of the spontaneous emission type photo conversion substance for light therapy according to this invention of FIG. 19, optical properties are 480 nm for blue and 530 nm for the excitation light emission peak, and each was verified in the light spectrum as 530, 560 and 565 nm for the photo conversion peak.

At which time, the particle size of the sapphire-based blue axial light fluorescent substance is 20.4 μm based on D50, and 2.18, 5.12, 4.65 μm based on D50 of the green/yellow conversion substance of M₂SiO₄:Eu²⁺ (M=Ba,Sr,Ca). Furthermore, the particle sizes of the final spontaneous emission type photo conversion substance for light therapy were 21.1, 2.22, and 20.8 μm based on D50.

### Preparing Example 4

The spontaneous emission type photo conversion substance for light therapy prepared through the method of preparing the spontaneous emission type photo conversion substance for light therapy of this invention was prepared by mixing at the weight ratio of “1” for the sapphire blue axial light fluorescent substance to “3” for the yellow conversion substance.

Consequently, as illustrated in the light spectrum of the preparing example 4 according to the preparing method of the spontaneous emission type photo conversion substance for light therapy according to this invention of FIG. 20, optical properties are 480 nm for blue and 545 nm for the excitation light emission peak and yellow, where the photo conversion peak was verified in the light spectrum.

At which time, the particle size of the sapphire-based blue axial light fluorescent substance is 20.4 μm based on D50, and 5.82 μm based on D50 of La₃Si₅N₁₁:Ce³⁺ yellow conversion substance. Furthermore, the particle size of the final spontaneous emission type photo conversion substance for light therapy was 25.8 μm based on D50.

### Preparing Example 5

The spontaneous emission type photo conversion substance for light therapy prepared through the method of preparing the spontaneous emission type photo conversion substance for light therapy of this invention was prepared by mixing at the weight ratio of “1” for the sapphire blue axial light fluorescent substance to “3” for the green/yellow conversion substance.

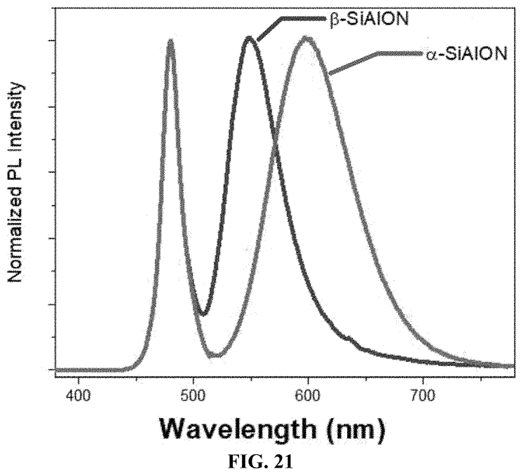

Consequently, as illustrated in the light spectrum of the preparing example 5 according to the preparing method of the spontaneous emission type photo conversion substance for light therapy according to this invention of FIG. 21, the optical properties are 480 nm for blue and 530 nm for the excitation light emission peak and green, and 590 nm for yellow, and the photo conversion peak was verified in the light spectrum.

At which time, the particle size of the sapphire-based blue axial light fluorescent substance is 20.4 μm based on D50, and 4.12 μm based on D50 of α-SiAlON and β-SiAlON green/yellow conversion substances. Furthermore, the particle size of the final spontaneous emission type photo conversion substance for light therapy was 28.1 μm based on D50.

### Preparing Example 6

The spontaneous emission type photo conversion substance for light therapy prepared through the method of preparing the spontaneous emission type photo conversion substance for light therapy of this invention was prepared by mixing at the weight ratio of “1” for the sapphire-based blue axial light fluorescent substance to “4” for the red conversion substance.

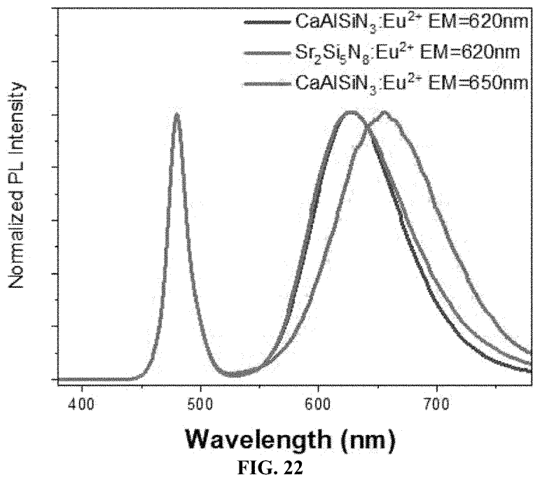

Consequently, as illustrated in the light spectrum of the preparing example 6 according to the preparing method of the spontaneous emission type photo conversion substance for light therapy according to this invention of FIG. 22, the optical properties are 480 nm for blue and 620 nm and 650 nm for the excitation light emission peak and red, and the photo conversion peak was verified in the light spectrum.

At which time, the particle size of the sapphire-based blue axial light fluorescent substance is 20.4 μm based on D50, and is 6.3 μm based on D50 of CaAlSiN₃:Eu³⁺/Sr₂Si₅N₈:Eu²⁺ red conversion substance. Furthermore, the particle size of the final spontaneous emission type photo conversion substance for light therapy was 28.5 μm based on D50.

### Preparing Example 7

The spontaneous emission type photo conversion substance for light therapy prepared through the method of preparing the spontaneous emission type photo conversion substance for light therapy of this invention was prepared by mixing at the weight ratio of “1” for the sapphire-based blue axial light fluorescent substance to “5” for the red conversion substance.

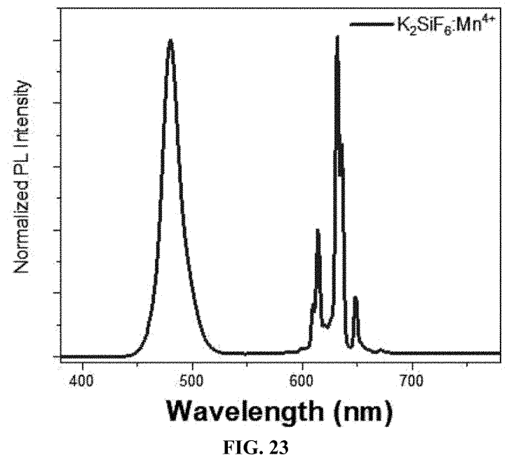

Consequently, as illustrated in the light spectrum of the preparing example 7 according to the preparing method of the spontaneous emission type photo conversion substance for light therapy according to this invention of FIG. 23, the optical properties are 480 nm for blue and 630 nm for the excitation light emission peak and red, and the photo conversion peak was verified in the light spectrum.

At which time, the particle size of the sapphire-based blue axial light fluorescent substance is 20.4 μm based on D50, and 3.2 μm based on D50 of K₂SiF₆:Mn⁴⁺ red conversion substance. Furthermore, the particle size of the final spontaneous emission type photo conversion substance for light therapy was 21.2 μm based on D50.

### Preparing Example 8

The spontaneous emission type photo conversion substance for light therapy prepared through the method of preparing the spontaneous emission type photo conversion substance for light therapy of this invention was prepared by mixing at the weight ratio of “1” for the sapphire-based blue axial light fluorescent substance to “4” for the red conversion substance.

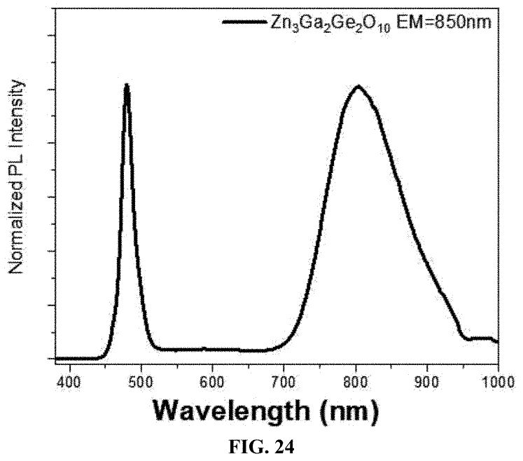

Consequently, as illustrated in the light spectrum of the preparing example 8 according to the preparing method of the spontaneous emission type photo conversion substance for light therapy according to this invention of FIG. 24, the optical properties are 480 nm for blue and 850 nm for the excitation light emission peak and red, and the photo conversion peak was verified in the light spectrum.

At which time, the particle size of the sapphire-based blue axial light fluorescent substance is 20.4 μm based on D50, and 1.2 μm based on D50 of Zn₃Ga₂Ge₂O₁₂ near infrared ray conversion substance. Furthermore, the particle size of the final spontaneous emission type photo conversion substance for light therapy was 20.9 μm based on D50.

### Implementation Example 1

The spontaneous emission type photo conversion substance for light therapy prepared in the preparing example 3 above was applied to a side of the TENCEL fabric by the printing method to prepare a sample having a means of light emission.

### Experimental Example 1

To verify the sterilization power, which is a characteristic of a 485 nm wavelength, concerning the sample prepared in the implementation example 1, for the excitation of the blue axial light fluorescent substance which forms the means of light emission of the sample prepared in the implementation example 1 (hereinafter, experimental group, a), after axial light was operated for 10 minutes every 6 hours by using an average white LED light (lantern), a microorganism culture solution was applied to the experimental group to verify the change of the microorganism.

Furthermore, a TENCEL fabric in which no means of light emission was formed is set as the control group (b), and an average white LED light (lantern) is used for 10 minutes to achieve the same conditions as the experimental group (a), after which the microorganism culture solution above was applied to verify the change of the microorganism.

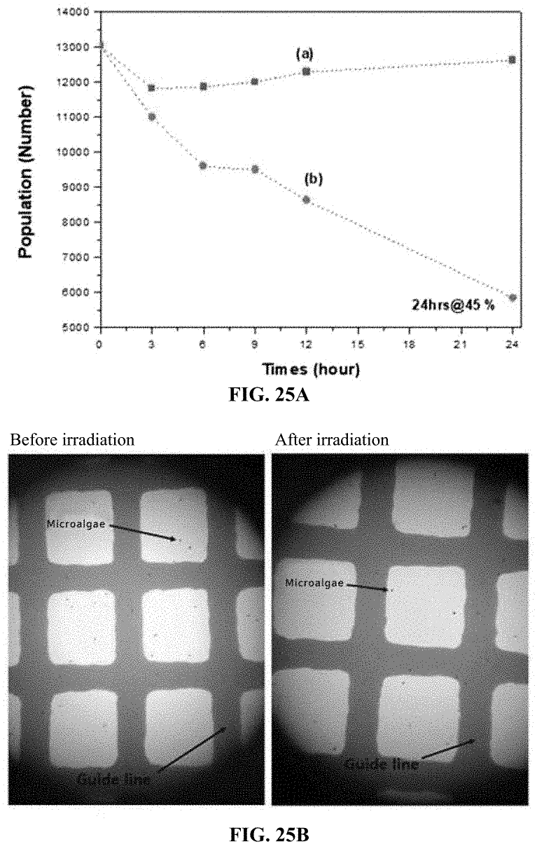

At which time, as for the microorganism above, Alexandrium A. Insuetum, which is a microalgal phytoplankton, was used, and the number of microalgae was counted every 3 hours from 00:00 to 24:00 hours, and as for FIGS. 25A-25B, as illustrated in the sterilization power comparison testing drawing of the implementation example 1 where the means of light emission comprising the spontaneous emission type photo conversion substance for light therapy according to this invention was formed, whereby the change was verified through representation in a spectrum.

As a result of the experiment, it was verified that the control group (b) did not have a significant change in the 13,000 value, whereas the experimental group (a) above continuously decreased from the 13,000 value until 24:00 hours and decreased down to 55%.

That is, it was verified that the population of the control group (b) made only of the TENCEL fabric was maintained on an average, and that there was no sterilization power. Furthermore, in the case of the number of individuals in the experimental group (a) where the means of light emission comprising the spontaneous emission type photo conversion substance for light therapy was formed, the sterilization efficiency was measured for 3, 6, 9, 12, and 24 hours based on the sterilization time, and consequently, it was verified that it was sterilized at approximately by 16%, 27%, 29%, 33.9%, and 55%, each respectively.

### Experimental Example 2

To verify the skin regeneration and improvement effect on the sample prepared in the implementation example 1, the sample prepared in the implementation example 1 was processed in a patch form and was attached to the same body part for 4 weeks for 30 minutes per day, after which changes in the skin density and the changes in the skin pigmentation were verified.

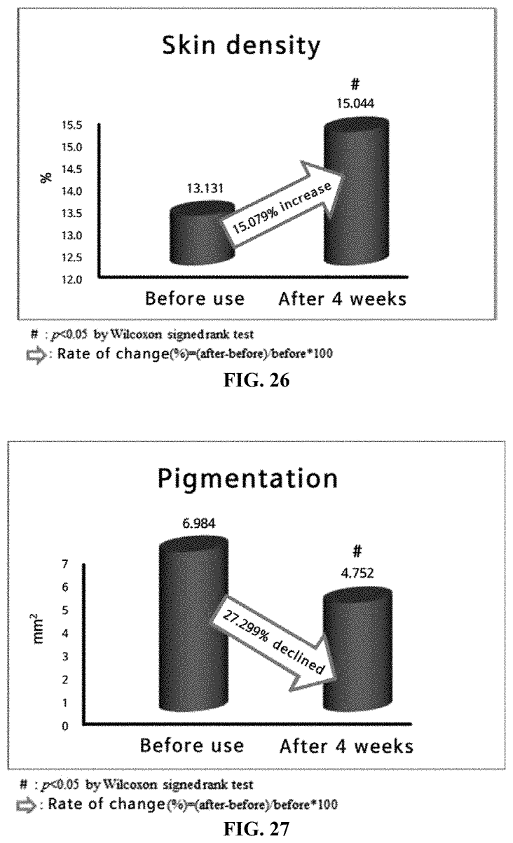

As illustrated in the results graph of the skin density test of the implementation example 1 where the means of light emission comprising the spontaneous emission type photo conversion substance for light therapy according to this invention was formed according to this invention of FIG. 26, the spontaneous emission type photo conversion substance for light therapy, as a result of using the implementation example 1 above where the means of light emission was formed for 4 weeks, it was verified that it has a significant effect on the skin regeneration as the skin density increased by 15% through the red and infrared rays.

Furthermore, as illustrated in the results graph of the pigmentation experiment of the implementation example 1 above where the means of light emission comprising the spontaneous emission type photo conversion substance for light therapy according to this invention was formed of FIG. 27, as a result of using the implementation example 1 above where the means of light emission comprising the spontaneous emission type photo conversion substance for light therapy was formed for 4 weeks, it was verified that there is an improvement effect of reducing 273% of wounds or skin pigmentation through the red light therapy.

While the implementation example of this invention has been described by making reference to the above and the attached drawings, those having conventional knowledge of the technical area to which this invention belongs can understand that this invention can be implemented in other specific forms without changing the technical ideas or essential features. Hence, it must be understood that the implementation examples described in the above are illustrative in all respects and are not restrictive.

1. A spontaneous emission type photo conversion substance for light therapy, comprising a blue light exciting core and a photo conversion shell enveloping the blue light exciting core,
wherein the blue light exciting core comprises a sapphire-based blue axial light fluorescent substance, wherein the sapphire-based blue axial light fluorescent substance is formed by mixing raw materials of inorganic oxides at molar equivalence, wherein the inorganic oxides have a chemical formula of Ma Al₂O₃, wherein M is at least one selected from the group consisting of Ba, Sr, Ca, Mg, Eu, and Dy;
wherein the sapphire-based blue axial light fluorescent substance is configured to output a prescribed wavelength according to a function for light therapy by delaying a light emission time with a surface defect treatment conducted via burning and grinding;
wherein the photo conversion shell comprises a photo conversion substance, wherein the photo conversion substance is prepared by mixing photo conversion raw materials with an alcohol and silicon nitride balls at a ratio of 1:3:2, wherein the photo conversion raw materials are at least one selected from the group consisting of a green conversion substance, a yellow conversion substance, a red conversion substance, and a near infrared ray conversion substance.

2. The spontaneous emission type photo conversion substance for light therapy of claim 1, wherein 0≤a<1.

3. The spontaneous emission type photo conversion substance for light therapy of claim 1, wherein the sapphire-base blue axial light fluorescent substance is formed by adding the europium (Eu) or the dysprosium (Dy) or the calcium (Ca) or the strontium (Sr) or the barium (Ba) or the magnesium (Mg) as a matrix to Al₂O₃, wherein the Al₂O₃ corresponds to aluminate and sapphire.

4. The spontaneous emission type photo conversion substance for light therapy of claim 1, wherein the green conversion substance and the yellow conversion substance are selected from the group consisting of YAG (Y₃Al₅O₁₂:Ce), LuAG (Lu₃Al₅O₁₂:Ce), M=(Ca,Sr,Ba), M₂SiO₄:Eu, M₃SiO₅:Eu, MSi₂O₂N₂:Eu, α-SiAlON, and β-SiAlON.

5. The spontaneous emission type photo conversion substance for light therapy of claim 1, wherein the red conversion substance is selected from the group consisting of M=(Ca,Sr,Ba), MAlSiN₃:Eu, and M₂Si₅N₈, K₂SiF₆:Mn.

6. The spontaneous emission type photo conversion substance for light therapy of claim 1, wherein the near infrared ray conversion substance comprises zinc and gallium and scandium oxides and a transition element, wherein the transition element is selected from the group consisting of manganese, chromium, and neodymium.

7. A functional patch, comprising the spontaneous emission type photo conversion substance of claim 1, a means of light emission, and a patch main unit having an adhesive material,
wherein the adhesive material is applied in a rear surface of the patch main unit and attached to various parts of a body of a user, the adhesive material has various sizes and shapes; and
the means of light emission is formed by a printing method in the rear surface of the patch main unit in contact with the body of the user.

8. The functional patch of claim 7, further comprising an adhesive fabric to maintain an attaching state of the patch main unit, wherein the adhesive fabric is formed of a same material as the patch main unit, wherein the adhesive fabric is wider than the patch main unit, the adhesive material is applied on a side of the adhesive fabric, and when the patch main unit attached to the body of the user, the adhesive fabric is attached to the body of the user while covering the patch main unit.

9. The functional patch of claim 7, wherein the means of light emission is a grid pattern printed across an entire rear surface of the patch main unit.

10. The functional patch of claim 9, wherein the grid pattern is made at an interval of 2 mm to 5 mm.

11. The functional patch of claim 7, wherein the patch main unit further comprises pharmaceutical ingredients.

12. A functional mask pack, comprising a mask sheet, the spontaneous emission type photo conversion substance of claim 1, and a mean of light emission, wherein
the mask sheet is immersed with a lotion and attached to a face of a user; and
the mean of light emission is formed by a printing method in an inner rear surface of the mask sheet, wherein the inner rear surface is in contact with the face of the user.

13. The functional mask pack of claim 12, wherein the means of light emission is printed in a patterned form designed to cover across an entirety of the inner rear surface of the mask sheet.

14. The functional mask pack of claim 13, wherein the means of light emission above is formed by being printed in a grid pattern across the entirety of the inner rear surface of the mask sheet.

15. The functional mask pack of claim 14, wherein the grid pattern is formed at an interval of 10 mm to 5 mm.

16. The functional mask pack of claim 12, wherein the mask sheet further comprises an extension unit, wherein the extension unit has a length and a width connected to a part of the mask sheet covering a forehead of the face and a head of the user.

17. The functional patch of claim 7, wherein 0≤a<1.

18. The functional patch of claim 7, wherein the sapphire-base blue axial light fluorescent substance is formed by adding the europium (Eu) or the dysprosium (Dy) or the calcium (Ca) or the strontium (Sr) or the barium (Ba) or the magnesium (Mg) as a matrix to Al₂O₃, wherein the Al₂O₃ corresponds to aluminate and sapphire.

19. The functional patch of claim 7, wherein the green conversion substance and the yellow conversion substance are selected from the group consisting of YAG (Y₃Al₅₀₁₂:Ce), LuAG (Lu₃Al₅O₁₂:Ce), M=(Ca,Sr,Ba), M₂SiO₄:Eu, M₃SiO₅:Eu, MSi₂O₂N₂:Eu, α-SiAlON, and β-SiAlON.

20. The functional patch of claim 7, wherein the red conversion substance is selected from the group consisting of M=(Ca,Sr,Ba), MAlSiN₃:Eu, and M₂Si₅N₈, K₂SiF₆:Mn.
